(19) United States
(12) Patent Application Publication
(10) Pub. No.: US 2015/0305716 A1
(43) Pub. Date: Oct. 29, 2015

(54) Ultrasound Transducer Array Apparatus and Method of Imaging Using Transducer Arrays

(71) Applicant: Koninklijke Philips N.V, Amsterdam (NL)

(72) Inventors: Cheryl D. Rice, San Diego, CA (US); David M. Sheehan, Poway, CA (US); Tom Esbeck, Carlsbad, CA (US)

(21) Appl. No.: 14/697,392

(22) Filed: Apr. 27, 2015

Related U.S. Application Data
(60) Provisional application No. 61/984,977, filed on Apr. 28, 2014.

(51) Int. Cl. A61B 8/00; A61B 8/12

(52) U.S. Cl. CPC A61B 8/4494; A61B 8/4461; A61B 8/445; A61B 8/12; A61B 8/461

(57) ABSTRACT

Solid-state ultrasound imaging devices, systems, and methods are provided. Some embodiments of the present disclosure are particularly directed to transducer arrays that employ a relatively large number of transducers yet are configured for fitting within space constraints associated with lumen and/or catheter dimensions. In one embodiment, an ultrasound imaging device includes a flexible elongate member; and an ultrasound scanner assembly disposed at a distal portion of the flexible elongate member, wherein the ultrasound scanner assembly includes an ultrasound transducer array, wherein the ultrasound transducer array includes a plurality of rings of transducers. In one aspect, the transducer arrays may be oscillated between multiple positions along the length of the flexible elongate member.

### CROSS REFERENCE TO RELATED APPLICATIONS

The present application claims priority to and the benefit of the U.S. Provisional Patent Application No. 61/984,977, filed Apr. 28, 2014, which is hereby incorporated by reference in its entirety.

### TECHNICAL FIELD

The present disclosure relates generally to ultrasound imaging and, in particular, to a solid-state intravascular ultrasound (IVUS) imaging system. In various embodiments, the imaging system includes an array of ultrasound transducers comprising a plurality of groupings of transducers, with each grouping of transducers circumferentially distributed about a longitudinal axis and different groupings distributed longitudinally with respect to the axis. Various embodiments include an actuator positioned to generate motion of the array. Various embodiments further include an outer catheter with the array positioned within the outer catheter. Embodiments presented herein are particularly well suited to imaging a human blood vessel.

### BACKGROUND

Intravascular ultrasound (IVUS) imaging is widely used in interventional cardiology as a diagnostic tool for a diseased vessel, such as an artery, within the human body to determine the need for treatment, to guide the intervention, and/or to assess its effectiveness. To perform an IVUS imaging study, an IVUS catheter that incorporates one or more ultrasound transducers is passed into the vessel and guided to the area to be imaged. The transducers emit and receive ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by one or more transducers and passed along to an IVUS imaging system, which is connected to the IVUS catheter by way of a patient interface module (PIM). The imaging system processes the received ultrasound signals to produce a cross-sectional image of the vessel where the device is placed.

One type of IVUS catheter commonly in use today is a solid-state IVUS catheter. Solid-state IVUS catheters carry an ultrasound scanner assembly that includes an array of ultrasound transducers distributed around the circumference of the device connected to a set of transducer control circuits. The transducer control circuits select individual transducers for transmitting an ultrasound pulse and for receiving the echo signal. By stepping through a sequence of transmitter-receiver pairs, the solid-state IVUS system can assemble a two-dimensional display of the vessel cross-section.

IVUS catheter performance depends on the quality of echo data generated by transducers. Solid-state IVUS catheters generate echo data typically using a single array of transducers distributed circumferentially about a longitudinal axis of a catheter assembly and aligned longitudinally.

While existing IVUS imaging systems have proved useful, there remains a need for improvements in imaging performance of solid-state IVUS catheters, for example, to provide improved accuracy and/or clarity of images. One way to improve imaging performance is increasing available echo data and utilizing increased echo data in image processing to provide higher-quality images. Accordingly, there is a persistent need for improvements to transducer arrays and associated mechanisms to take advantage of improved transducer arrays that could lead to improvements in imaging performance.

### SUMMARY

Embodiments of the present disclosure provide transducer arrays in solid-state ultrasound imaging systems that provide for improved imaging of vessels. Embodiments additionally provide transducer arrays in movable configurations, actuator mechanisms for moving the arrays, and outer catheters for covering the arrays, all in a solid-state imaging system, that together provide for improved imaging of vessels.

In some embodiments, an intravascular ultrasound (IVUS) device is provided. The device comprises a flexible elongate member; and an ultrasound scanner assembly disposed at a distal portion of the flexible elongate member, wherein the ultrasound scanner assembly includes an ultrasound transducer array, wherein the ultrasound transducer array includes a plurality of rings of transducers.

In some embodiments, an IVUS device is provided. The device comprises a flexible elongate member; and an ultrasound scanner assembly disposed at a distal portion of the flexible elongate member; wherein the ultrasound scanner assembly includes: an actuator mechanism comprising an actuator; a longitudinal ultrasound transducer array; and a control circuit positioned between the actuator and the transducer array, wherein the control circuit and the transducer array are configured to move in response to movement of the actuator.

In some embodiments, a method of ultrasound imaging using an IVUS device is provided, wherein the device comprises an ultrasound scanner assembly including an ultrasound transducer array, and wherein the ultrasound transducer array includes a plurality of rings of transducers. The method comprises: for each of the plurality of rings, performing the following: emitting an ultrasonic waveform by at least one transducer in the corresponding ring; generating echo data by the ultrasound scanner assembly based on a reflected echo of the ultrasonic waveform; and providing the echo data to an IVUS console. The method further comprises processing the echo data generated by each of the plurality of rings for display.

Some embodiments of the present disclosure utilize a longitudinal transducer array comprising a plurality of circumferential arrays of transducers. Each circumferential array comprises a corresponding plurality of transducers. A circumferential array can comprise 16, 32, or 64 transducers as illustrative but non-limiting examples, and a longitudinal transducer array can comprise two, three, or four circumferential arrays as illustrative but non-limiting examples. Each circumferential array of the longitudinal array can emit ultrasonic signals and the transducer array can be employed to receive echo signals. In some embodiments, an actuator is coupled to the transducer array to provide motion, resulting in, for example, longitudinal oscillation of the transducer array. By coordinating the firing of circumferential arrays with movement of the actuator, the firing position of each circumferential array is controlled. For example, each circumferential array can be fired in substantially the same longitudinal position and software can be employed to perform an average or weighted average of the echo signals to generate an image of blood vessels for display. In some embodiments, an outer catheter is employed to protect blood vessels from movement of the transducer array. In some embodiments, the outer catheter is a continuous outer catheter and in other embodiments the outer catheter has openings that are transparent to ultrasonic signals.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an imaging system according to an embodiment of the present disclosure.

FIG. 2 is a top view of an ultrasound transducer array depicted in its flat form according to an embodiment of the present disclosure.

FIG. 3 is a longitudinal perspective view of an ultrasound transducer array according to an embodiment of the present disclosure.

FIG. 4 is a top view of an ultrasound transducer array depicted in its flat form according to an embodiment of the present disclosure.

FIG. 5 is a top view of a portion of an ultrasound scanner assembly depicted in its flat form according to an embodiment of the present disclosure.

FIG. 6 is a longitudinal perspective view of a portion of an ultrasound scanner assembly depicted in its rolled form according to an embodiment of the present disclosure.

FIG. 7 is a partial cut-away perspective view of an ultrasound scanner assembly according to an embodiment of the present disclosure.

FIGS. 8A-8C are top views of an ultrasound transducer array depicted in its flat form depicted at various combinations of position and firing configuration according to an embodiment of the present disclosure.

FIG. 9 is a side view of a portion of an outer catheter according to an embodiment of the present disclosure.

FIG. 10 is a side view of a portion of an outer catheter according to an embodiment of the present disclosure.

FIG. 11 is a longitudinal perspective view of an ultrasound transducer array and an outer catheter according to an embodiment of the present disclosure.

FIGS. 12A-12C are top views of ultrasound transducer arrays depicted in their flat form according to various embodiments of the present disclosure.

FIGS. 13A and 13B are cross-sectional views of transducer regions of ultrasound scanner assemblies according to embodiments of the present disclosure

FIG. 14 is a flow diagram of a method of generating ultrasound data according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the IVUS system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

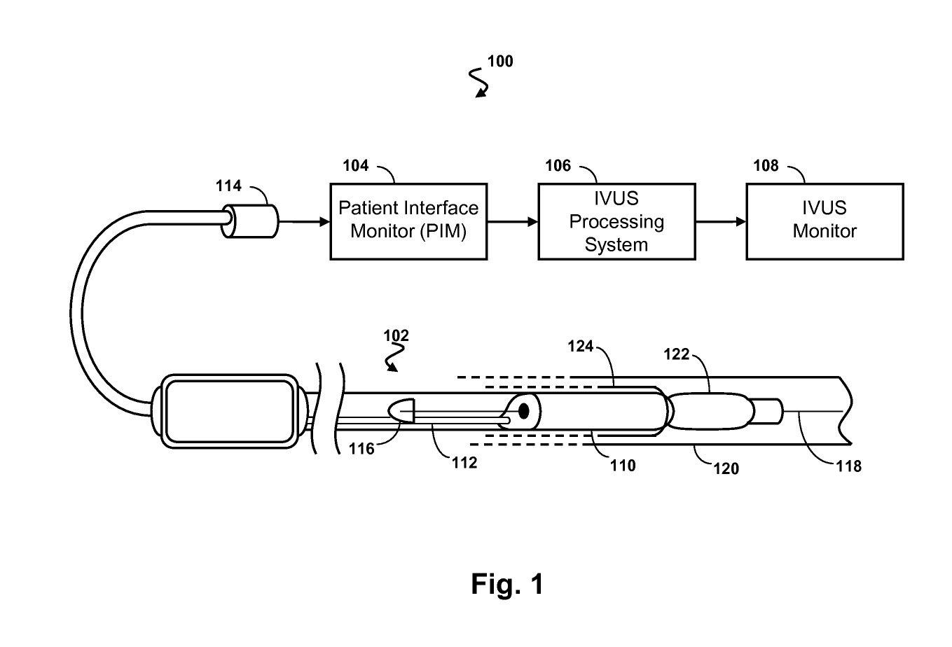

FIG. 1 is a diagrammatic schematic view of an ultrasound imaging system 100 according to an embodiment of the present disclosure. In some embodiments of the present disclosure, the imaging system 100 is a piezoelectric zirconate transducer (PZT) solid-state imaging system. In some embodiments, the system 100 incorporates capacitive micromachined ultrasonic transducers (CMUTs), and/or piezoelectric micromachined ultrasound transducers (PMUTs). The imaging system 100 may be utilized for intravascular ultrasound (IVUS) imaging and may include an IVUS catheter or device 102 such as a catheter, guide wire, or guide catheter, a patient interface module (PIM) 104, an IVUS console or processing system 106, and/or a monitor 108.

At a high level, the IVUS device 102 emits ultrasonic energy from a scanner assembly 110 at the tip of the device. The ultrasonic energy is reflected by tissue structures surrounding the scanner 110 and the echo signals from the tissue are received and amplified by the scanner 110.

The PIM 104 facilitates communication of signals between the IVUS console 106 and the IVUS device 102 to control the operation of the scanner assembly 110. This includes generating control signals to configure the scanner and trigger the transmitter circuits and transferring echo signals captured by the scanner assembly 110 to the IUVS console 106. With regard to the echo signals, the PIM 104 forwards the received signals and, in some embodiments, performs preliminary signal processing prior to transmitting the signals to the console 106. In examples of such embodiments, the PIM 104 performs amplification, filtering, and/or aggregating of the data. In an embodiment, the PIM 104 also supplies high- and low-voltage DC power to support operation of the circuitry within the scanner 110.

The IVUS console 106 receives the echo data from the scanner 110 by way of the PIM 104 and processes the data to create an image of the tissue surrounding the scanner 110. The console 106 may also display the image on the monitor 108.

In some embodiments, the IVUS device 102 includes some features similar to traditional solid-state IVUS catheters, such as the EagleEye® catheter available from Volcano Corporation and those disclosed in U.S. Pat. No. 7,846,101 hereby incorporated by reference in its entirety. For example, the IVUS device 102 includes the ultrasound scanner assembly 110 at a distal end of the device 102 and a cable 112 extending along the longitudinal body of the device 102. The cable 112 terminates in a connector 114 at a proximal end of the device 102. The connector 114 electrically couples the cable 112 to the PIM 104 and physically couples the IVUS device 102 to the PIM 104.

In some embodiments, an outer catheter 124 circumscribes at least a portion of the IVUS device 102, with at least a portion of the ultrasound scanner assembly 110 located inside the outer catheter 124. In an embodiment, a longitudinal axis of the outer catheter 124 is substantially aligned with a longitudinal axis of the ultrasound scanner assembly 110 and the IVUS device 102. One function of the outer catheter 124 is to protect the vessel 120 from movement of a portion of the scanner assembly 110 as described more fully below.

In an embodiment, the IVUS device 102 further includes a guide wire exit port 116. Accordingly, in some instances the IVUS device 102 is a rapid-exchange catheter. The guide wire exit port 116 allows a guide wire 118 to be inserted towards the distal end of the device 102 in order to direct the device 102 through a vessel 120. Vessel 120 represents fluid filled or surrounded structures including arteries and veins, both natural and man-made, within a living body that may be imaged and can include for example, but without limitation, structures such as: organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body. In addition to imaging natural structures, the images may also include imaging man-made structures such as, but without limitation, heart valves, stents, shunts, filters and other devices positioned within the body.

In an embodiment, the IVUS device 102 also includes an inflatable balloon portion 122 near the distal tip. The balloon portion 122 is open to a lumen that travels along the length of the IVUS catheter and ends in an inflation port (not shown). The balloon 122 may be selectively inflated and deflated via the inflation port. The IVUS device 102 has a diameter of between about 2.9 French (F) and about 5 F. Thus, in one example, the balloon and imaging array may be used for the placement of treating structures, such as stents or drug eluting coatings, in the vessels being imaged. In still a further form, the imaging array described herein can be utilized to place and retrieve intravascular filters.

The IVUS device 102 is designed to provide high-resolution imaging from within narrow passageways. To advance the performance of IVUS imaging devices compared to the current state of the art, embodiments of the present disclosure can incorporate advanced transducer technologies, such as PMUT, CMUT, single crystal or other devices that offer wide bandwidth (>100%). The broad bandwidth is important for producing a short ultrasound pulse to achieve optimum resolution in the radial direction. The improved resolution provided by PMUT and other advanced ultrasound transducer technologies facilitates better diagnostic accuracy, enhances the ability to discern different tissue types, and enhances the ability to accurately ascertain the borders of the vessel lumen. Embodiments of the present disclosure have enhanced ability to generate, collect, and/or process ultrasonic echo data allowing for improved cardiovascular imaging.

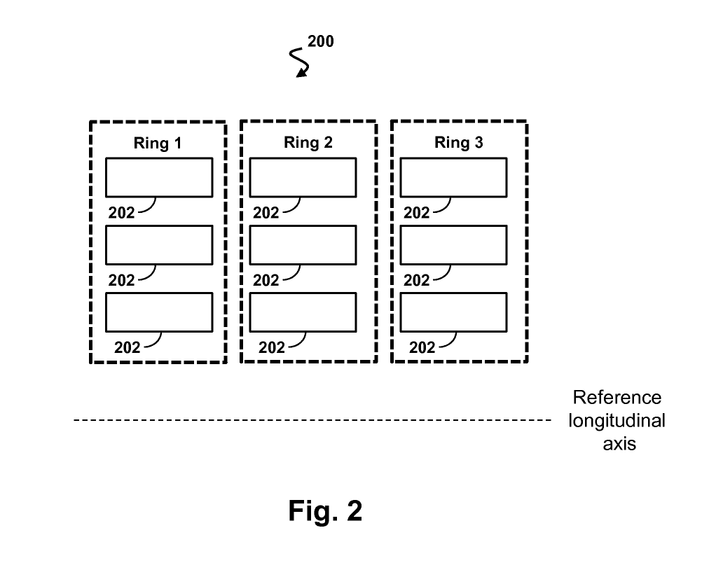

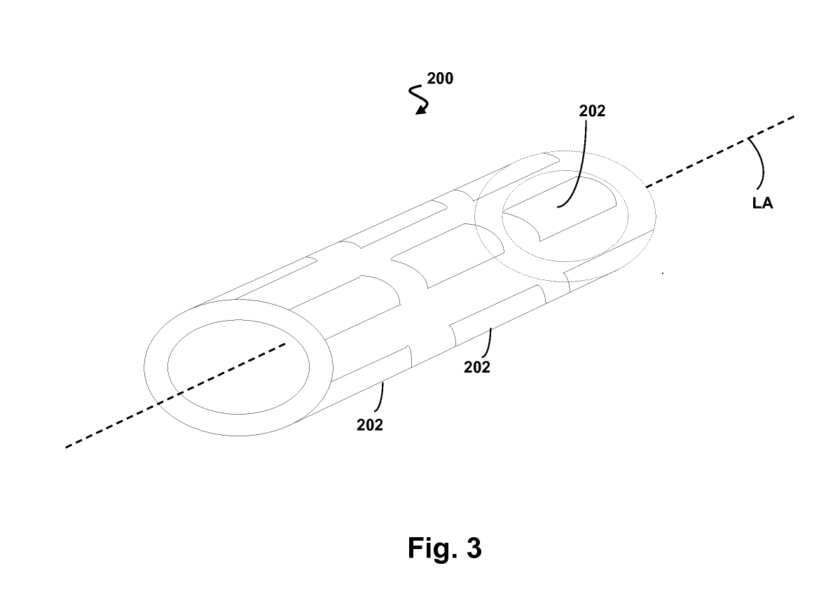

FIG. 2 is a top view of an ultrasound transducer array 200 depicted in its flat form according to an embodiment of the present disclosure. The transducer array 200 comprises a portion of the ultrasound scanner assembly 110 depicted in FIG. 1. In this example, the transducer array 200 comprises nine transducers 202 as shown for illustration, but the transducer array 200 can comprise any number of transducers 202. The transducer array 200 comprises three rings of transducers 202, each of which is placed circumferentially around a longitudinal axis of an ultrasound scanner assembly 110, as illustrated further in FIG. 3. An example reference longitudinal axis is illustrated. In the illustrated embodiment, rings could have a different number of individual transducers. Further, the number of transducers in each ring may be correlated with the frequency of each transducer ring. For example, there may be few transducers at a lower frequency while more transducers may be utilized at higher frequencies. Each ring of transducers 202 comprises a same number of transducers (in this example, three transducers 202), and the rings are in helical alignment in the sense that there is substantially no angular rotation of one ring with respect to another. In this example, a ring refers to a plurality of transducers distributed circumferentially about a longitudinal axis at substantially the same longitudinal position. In some embodiments, a transducer array comprising a plurality of circumferential arrays or rings of transducers may be referred to as a longitudinal array.

FIG. 3 is a longitudinal perspective view of an ultrasound transducer array 200 according to an embodiment of the present disclosure. The transducer array 200 is depicted in its rolled form. In that regard, in some instances the scanner is transitioned from a flat configuration to a rolled or more cylindrical configuration. The transducer array 200 comprises a portion of scanner assembly 110 as depicted in FIG. 1. A longitudinal axis (LA) of the transducer array 200 is aligned with or parallel to a longitudinal axis of the IVUS catheter 102.

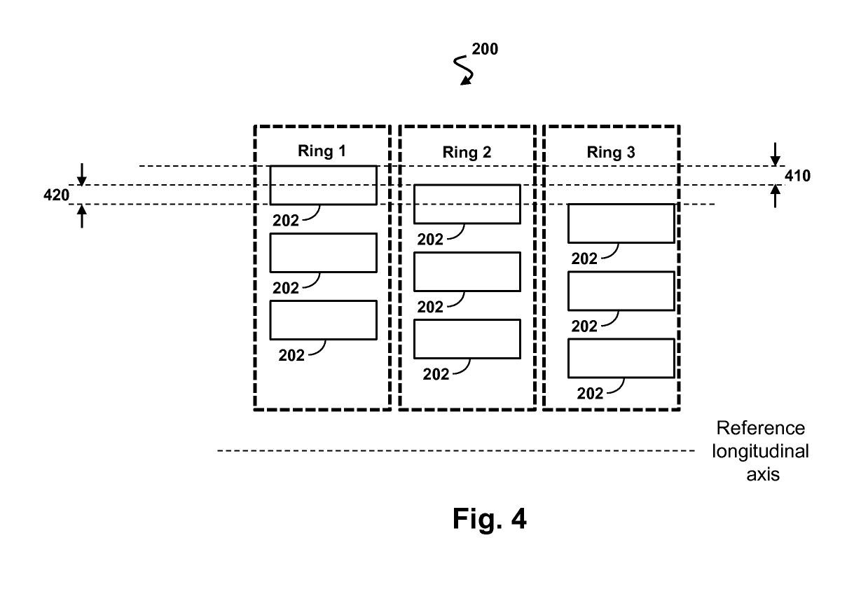

FIG. 4 is a top view of an ultrasound transducer array 200 depicted in its flat form according to an embodiment of the present disclosure. The transducer array 200 comprises a portion of the ultrasound scanner assembly 110 depicted in FIG. 1. Like the embodiment transducer array 200 in FIG. 2, the transducer array 200 in FIG. 4 comprises nine transducers divided into three rings, but unlike in FIG. 2 the rings are helically offset from one another. For example, as shown ring 2 is offset from ring 1 by an amount depicted as 410. Furthermore, ring 3 is offset from ring 2 by an amount depicted as 420. The offsets translate to rotational or helical offsets of one ring with respect to another ring when the transducer array 200 is produced in rolled form.

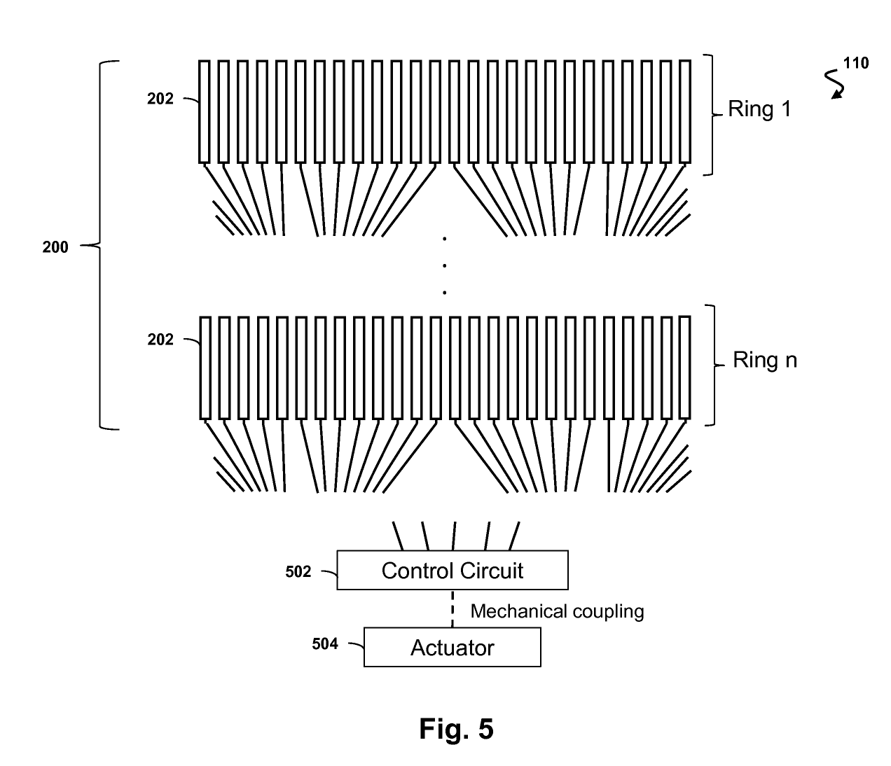

FIG. 5 is a top view of a portion of an ultrasound scanner assembly 110 depicted in its flat form according to an embodiment of the present disclosure. The assembly includes a transducer array 200, a control circuit 502, and an actuator 504. As indicated by the common reference numbers, the ultrasound transducers 202 of the transducer array 200 are substantially similar to those disclosed with reference to FIGS. 2-4. The transducer array 200 may include any number and type of ultrasound transducers 202, although for clarity only a limited number of ultrasound transducers are illustrated in FIG. 5. The transducer array 200 includes n rings (or circumferential arrays) of transducers 202, where n is an integer greater than one (e.g., n=two, three, or four). In an embodiment, each ring of transducers 202 includes 64 individual transducers 202. In another embodiment, each ring of transducers 202 includes 128 individual transducers. In another embodiment, each ring of transducers includes 32 ultrasound transducers 202. In another embodiment, each ring of transducers includes 16 ultrasound transducers 202. Although the illustrated embodiments show transducer arrays having as few as three transducer elements, it is contemplated that arrays can have 16 or more transducer elements, including configurations with 32 or 64 transducers. Other numbers are both contemplated and provided for.

In an embodiment, the ultrasound transducers 202 of the transducer array 200 include PZT transducers, such as bulk PZT transducers, capacitive micromachined ultrasound transducers (cMUTs), single crystal piezoelectric materials, other suitable ultrasound transmitters and receivers, and/or combinations thereof. In an alternative embodiment, the ultrasound transducers 202 of the transducer array 200 include PMUTs fabricated on a microelectromechanical system (MEMS) substrate using a polymer piezoelectric material, for example as disclosed in U.S. Pat. No. 6,641,540, which is hereby incorporated by reference in its entirety.

The control circuit 502 is electrically connected to each of the transducers 202. In an embodiment, the control circuit 502 is configured to drive each of the transducers 202 to generate ultrasound signals. The control circuit 502 also receives echo data from a number of transducers 202 and retransmits it on a cable (not shown). In some embodiments, the control circuit 502 receives unamplified or partially amplified echo data and performs amplification for driving the echo data along conductors of a cable. In some embodiments, the control circuit may comprise a master controller and slave controllers as described in U.S. patent application Ser. No. 14/137,269, published as U.S. Patent Application Publication No. 2014/0187960 on Jul. 3, 2014,” which is hereby incorporated by reference in its entirety.

The control circuit 502 causes the transducers 202 to emit signals in any pattern. For example, the control circuit 502 can cause each transducer 202 in a ring to fire or emit sequentially stepping through the transducers 202 in any order, such as starting with one transducer 202 and stepping through adjacent transducers 202 until all transducers 202 have fired. Then the control circuit 502 can cause the next ring of transducers 202 to fire in a similar manner. The pattern can be repeated until all transducers 202 have fired. Likewise, the control circuit 502 can receive echoes from any number of transducers 502 in response to any echo signal in any pattern.

As described in U.S. Patent Application Publication No. 2014/0187960 referred to above, in an embodiment, a flex circuit (not shown) provides structural support and physically connects the transducer control circuit 502 and the transducers 202. The flex circuit may contain a film layer of a flexible polyimide material such as KAPTON™ (trademark of DuPont). Other suitable materials include polyester films, polyimide films, polyethylene napthalate films, or polyetherimide films, other flexible printed circuit substrates as well as products such as Upilex® (registered trademark of Ube Industries) and TEFLON® (registered trademark of E.I. du Pont). The film layer is configured to be wrapped around a ferrule to form a cylindrical toroid in some instances. Therefore, the thickness of the film layer is generally related to the degree of curvature in the final assembled scanner 110.

The scanner assembly 110 includes an actuator mechanism 504 to move the scanner assembly longitudinally along the longitudinal axis. The actuator mechanism 504 comprises at least one anchor and at least one actuator. At least one anchor is secured to the device 102, and at least one actuator is mechanically coupled to the control circuit 502. In various embodiments, the control circuit 502 and the transducer array 200 move as a unit when driven by at least one actuator.

There are at least two modes of operation for which a transducer ring listens for echoes. In a first mode, the firing ring is also the listening ring. In this mode, a ring cannot move substantially in a time period equal to two times the maximum imaging distance divided by the speed of flight. In a second mode, a ring different than the firing ring is the listening ring. In the second mode, the array should move enough in a maximum round-trip echo time that another ring is in place for receiving. These constraints dictate the desired speed of longitudinal movement of a transducer array.

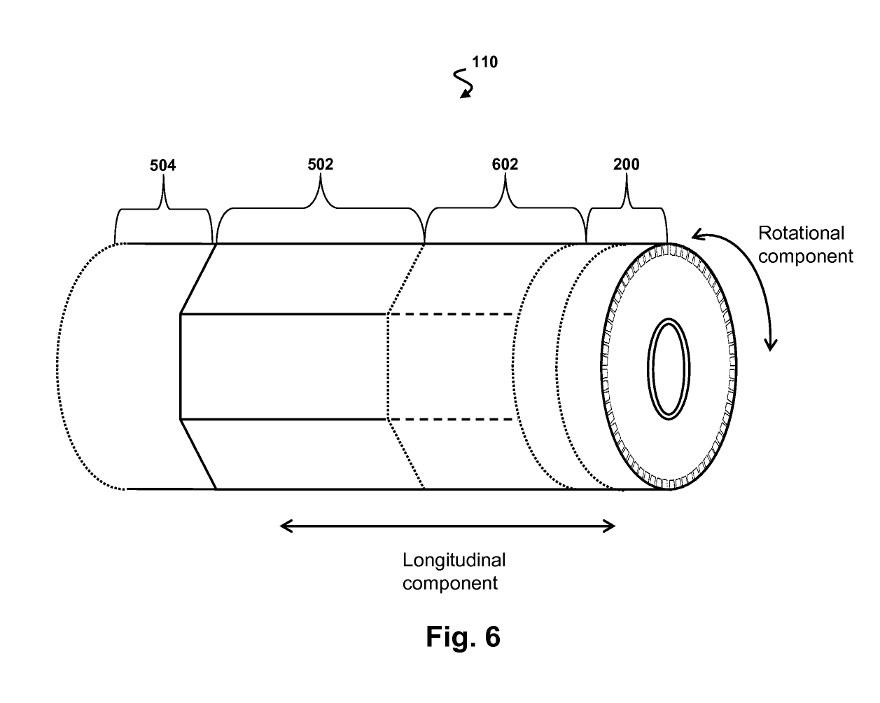

FIG. 6 is a longitudinal perspective view of a portion of an ultrasound scanner assembly 110 depicted in its rolled form according to an embodiment of the present disclosure. Referring to FIG. 6, a transition portion 602 is located between the transducer array 200 and the control circuit 502. As shown, the transducer array 200 comprises two rings of transducers, but as explained previously, the transducer array 200 can comprise any number of rings. The scanner assembly further comprises an actuator mechanism 504 positioned to move the control circuit 502, transition region 602, and transducer array 200 longitudinally along the longitudinal axis of the device. In various embodiments, the control circuit 502, transition region 602, and transducer array 200 move as a unit. The control circuit 502, transition region 602, and transducer array 200 can be considered as a movable portion of the scanner assembly 110. Possible directions of motion are shown in FIG. 6. As shown, control circuit 502, transition region 602, and transducer array 200 are capable of motion involving a combination of rotational component and/or a longitudinal component.

In contrast to the transducer array 200 and the control circuit 502, the transition portion 602 is free of rigid structures. Instead, the cross-sectional shape is defined by the adjacent regions 200 and 502. Thus, the shape of the transition portion 602 transitions between that of the transducer array 200 and the control circuit 502. The transition portion 602 may be used to reduce sharp angles that can stress the flex circuit and/or the conductive traces. Because of the more circular cross-section of the associated control circuit 502 of the present disclosure, embodiments utilizing, for example, 8, 9, 16, or more transducer control circuits support a shorter transition region 602. In other words, because both transducer control circuits and ultrasound transducers 202 produce flat areas within a flex circuit, substituting physically narrower devices reduces the noncircular regions of the flex circuit caused by each individual device.

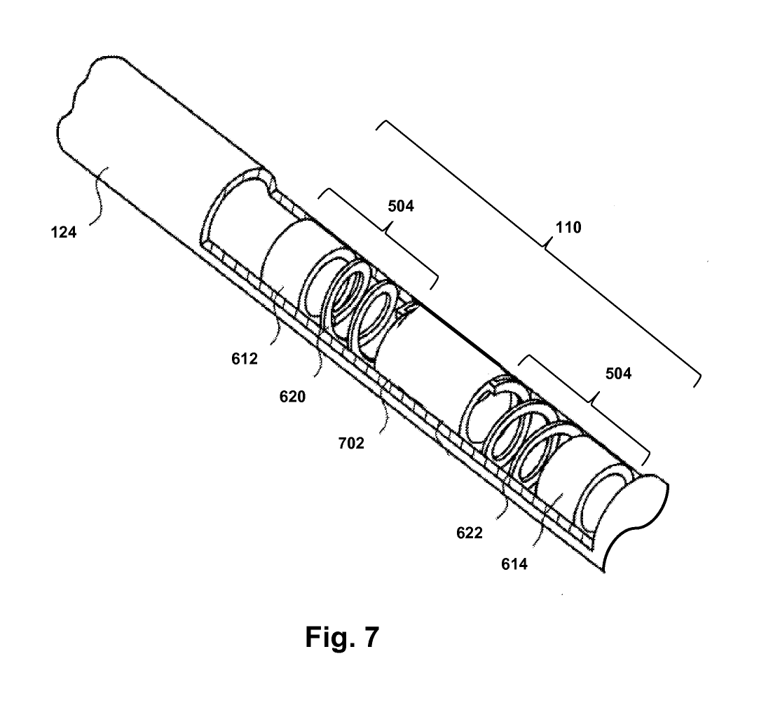

FIG. 7 is a partial cut-away perspective view of an ultrasound scanner assembly 110 according to an embodiment of the present disclosure. The ultrasound scanner assembly 110 comprises a pair of actuator mechanisms 504 and a movable element 702. In various embodiments, the movable element 702 comprises a transducer array 200, a control circuit 502, and a transition region 602. In various embodiments, the movable element 702 and the actuator mechanisms 504 are housed in the outer catheter 124 and are part of an IVUS device 102. Example embodiments of the outer catheter 124 are described more fully below.

FIG. 7 illustrates a scanner assembly 110, which is housed in the distal end of an elongate member, with the longitudinal axis of the scanner assembly 110 oriented substantially parallel to the longitudinal axis of outer catheter 124. The actuator mechanisms 504 include a first anchor 612 and a second anchor 614 which are secured relative to the transducer array 200 and control circuit 502 to anchor the actuator mechanisms 504 such that the anchors 612 and 614 cannot move relative to the movable element 702. The movable element 702 is not secured relative to the anchors 612 and 614 and is free to move in at least one range of motion relative to the anchors 612 and 614 and the outer catheter 124.

The first anchor 612 is connected to the movable element 702 by a shape memory alloy (SMA) actuator 620 which moves movable element 702 when activated as described in more detail below. The SMA actuator 620 can be fabricated from any known material with shape memory characteristics. In an alternative embodiment the actuator mechanisms 504 can be fabricated without from a single tubing using any material with shape memory characteristics, incorporating the first anchor 612, second anchor 614, moveable element 702, SMA actuator 620 and deformable component 622. As known by those of skill in the art, SMAs can be fabricated to take on a predetermined shape when activated. Activation of an SMA actuator consists of heating the SMA such that it adopts its trained shape. Typically, this is accomplished by applying an electric current across the SMA element. Deactivation of an SMA actuator includes turning off current to SMA, such that it returns to its pliable state as it cools. Activation of the SMA to its trained shape results in a force which can be utilized as an actuator. As one of skill in the art will recognize, the disclosed SMA actuator 20 can take numerous shapes and configurations in addition to the helical shape shown in FIG. 1. For example it could be linear, or more than one (e.g. 2, 3, 4 or more) SMA elements could be used to make the SMA actuator 20. Further detail regarding the actuator mechanisms 504 can be found in U.S. patent application Ser. No. 11/415,855, published as U.S. Patent Application Publication No. 2007/0016063 on Jan. 18, 2007, and incorporated herein by reference. The actuator mechanisms 504 are an example configuration for moving the movable element 702. Other potential actuator mechanisms are described in U.S. Patent Publication 2007/0016063 referenced above.

There are other potential locations for an actuator mechanism other than adjacent to the movable element 702. For example, an actuator mechanism can be placed at a proximal end of an elongate member so that the actuator mechanism is outside a human body. In various embodiments, the actuator mechanism is mechanically coupled to a scanner assembly via a structure, such as a catheter.

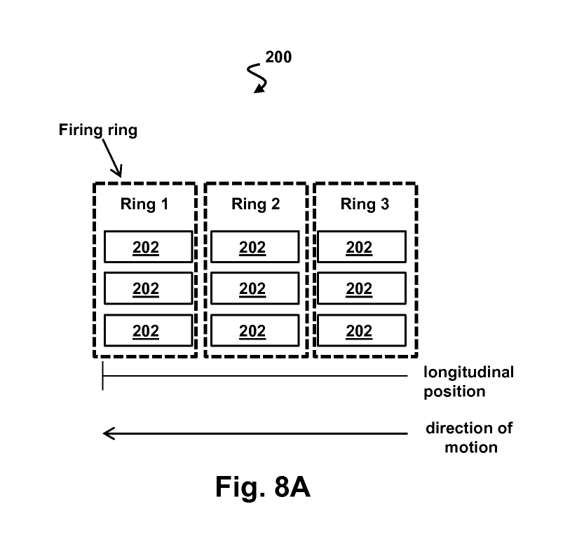

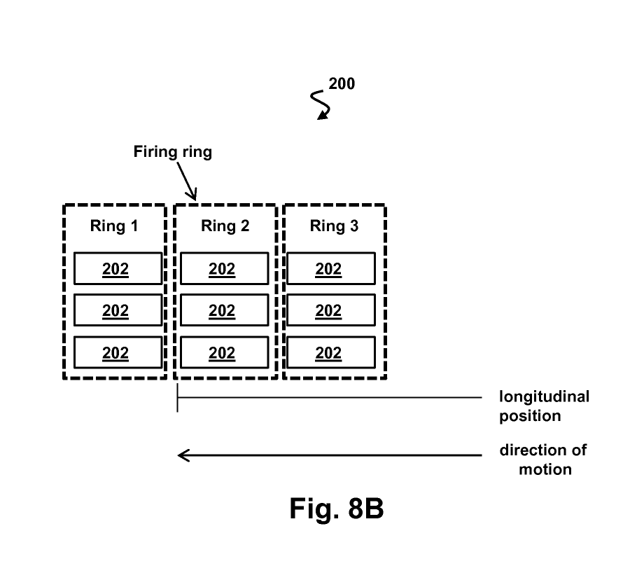

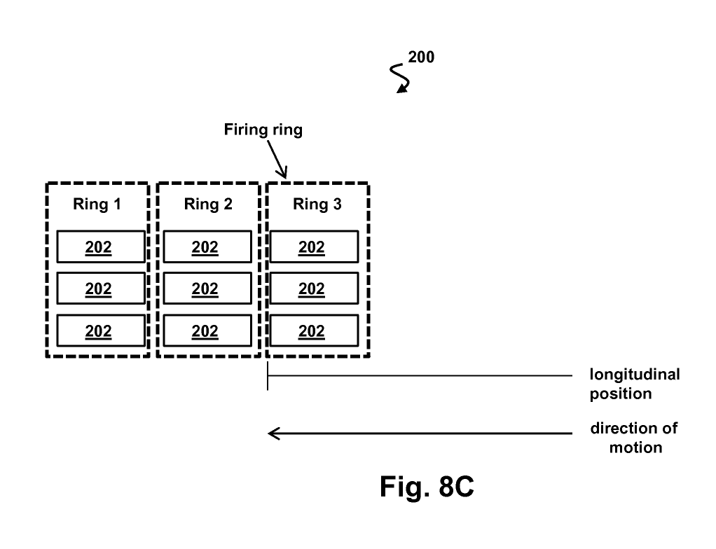

FIGS. 8A-8C are top views of an ultrasound transducer array 200 depicted in its flat form depicted at various combinations of position and firing configuration according to an embodiment of the present disclosure. FIGS. 8A-8C illustrate a transducer array 200 comprising nine transducers 202 organized into three rings. The direction of motion and position with respect to a fixed reference point, such as an actuator anchor or outer catheter, are illustrated. FIG. 8A illustrates the transducer array 200 at a first point in time and position. The direction of motion is illustrated. In FIG. 8A, the transducers 202 in the first ring are fired to emit ultrasonic signals. One or more transducers 202 in ring one are fired until all transducers in ring one have emitted an ultrasound signal.

FIG. 8B illustrates the transducer array 200 at a second point in time and a second position. In FIG. 8B, the transducers in the second ring are fired to emit ultrasonic signals. One or more transducers 202 in ring two are fired until all transducers in ring two have emitted an ultrasound signal.

FIG. 8C illustrates the transducer array 200 at a second point in time and a second position. In FIG. 8C, the transducers in the second ring are fired to emit ultrasonic signals. One or more transducers 202 in ring three are fired until all transducers in ring three have emitted an ultrasound signal.

The motion of the transducer array 200 may be continuous in moving between the positions illustrated in FIGS. 8A-8C. The speed of sequencing through firing of transducers in a ring may be such that a ring of transducers 202 is fired in substantially the same longitudinal position. By firing all three rings at the same or substantially the same longitudinal position as shown, data can be collected for a cross section of a blood vessel at the region of firing. Furthermore, after cycling through the positions illustrated in FIGS. 8A-8C, the transducer array 200 may then return to the starting position of FIG. 8A. In various embodiments, the transducer array 200 has an oscillating motion wherein the transducer array 200 oscillates back and forth along a longitudinal axis around which the transducer array 200 is folded. The speed of oscillation may be set to a desired speed for ease of processing. Alternatively, the speed of oscillation may be changed by the user to optimize the array for various vessel sizes or conditions. For example, for larger vessels the oscillation may be slowed to take into account the longer time of flight for the ultrasound echoes.

The embodiments in FIGS. 8A-8C are not restricted to rings that are in helical alignment. For example, the firing pattern illustrated in FIGS. 8A-8C can be applied to the embodiment of transducer arrays with helical offsets, such as the transducer array 200 illustrated in FIG. 4.

In various embodiments, a control circuit 502 is electrically coupled to the transducer array 200 to control firing of the transducers 202 and collection of echo data via the transducers 202. Furthermore, an actuator mechanism 504, as illustrated in various figures herein, effects motion of the transducer array 200 to move the actuator array 200 in the manner illustrated in FIGS. 8A-8C.

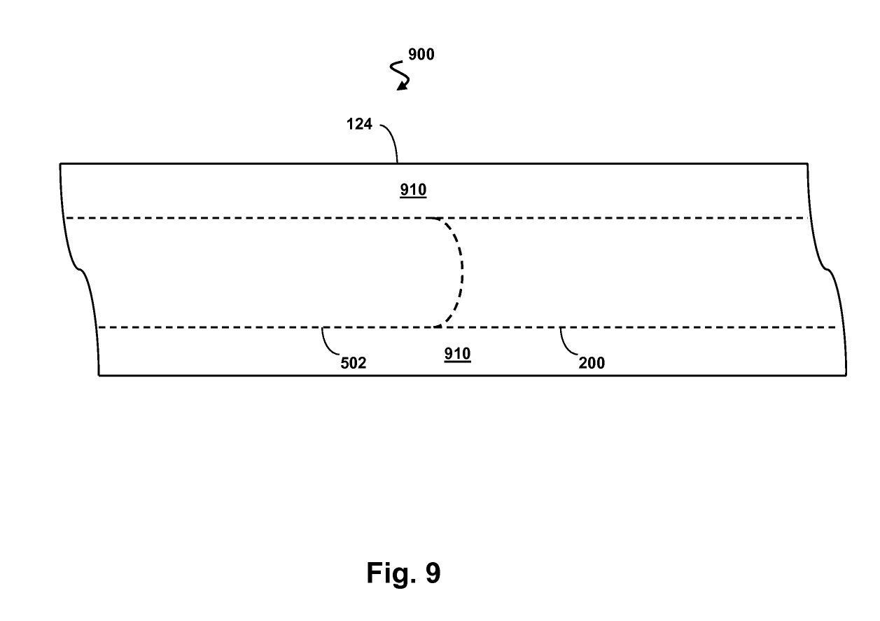

FIG. 9 is a side view of a region 900 of a continuous outer sheath or catheter according to an embodiment of the present disclosure. The region 900 illustrates a continuous outer catheter 124 surrounding a transducer array 200 and control circuit 502. As described previously, the control circuit 502 and transducer array are movable in some embodiments. A lubricious fluid 910 is disposed between the outer catheter 124 and the transducer array 200 and the control circuit 502 as shown. In this embodiment, the outer catheter 124 is continuous so that the lubricious fluid 910 does not mix with the blood. The fluid 910 acts as a lubricant between the outer catheter 124 and the transducer array 200. Furthermore, the lubricious fluid 910 can be impedance matched with the PZT transducer, along a matching layer overlaying the transducer if utilized, or the outer catheter 124 for ultrasound transmission from the transducer array 200 into the blood. In one form, the transducer is utilized without a matching layer and the lubricious fluid 910 is selected to impedance match the transducer to the catheter. In this configuration, the fluid 910 is utilized as the impedance matching layer. FIG. 9 is for illustrative purposes only and not drawn to scale. For example, a thickness of the outer catheter 124 is not shown, but the outer catheter 124 will in practice have a thickness. In an embodiment, a longitudinal axis of the outer catheter 124 is substantially aligned with a longitudinal axis of the transducer array 200.

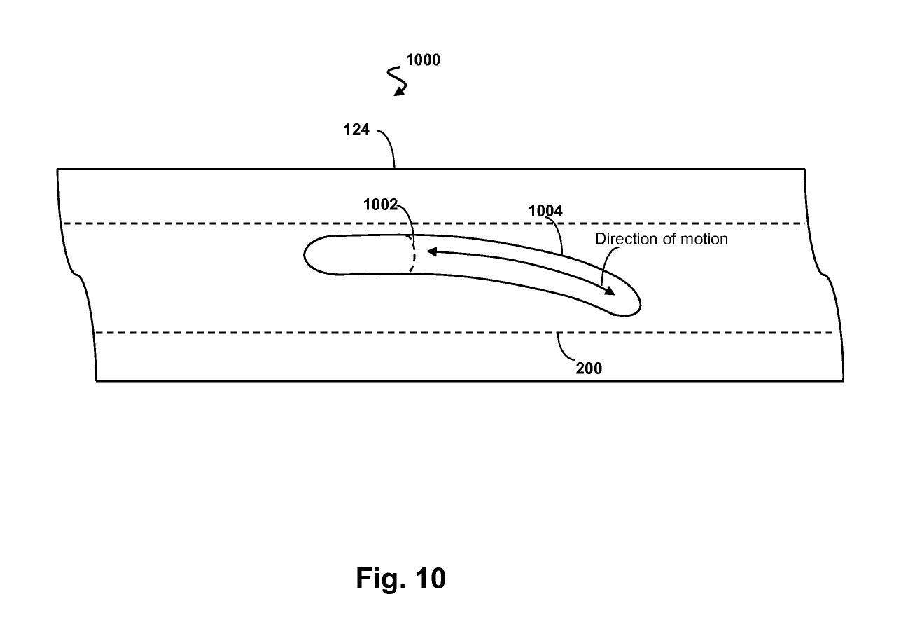

FIG. 10 is a side view of a region 1000 of an outer catheter according to an embodiment of the present disclosure. The region 1000 comprises an outer catheter 124 circumscribing or surrounding a transducer array 200. In this embodiment, the outer catheter 124 includes a guide 1004 and the transducer array 200 includes a notch 1002 that fits inside the guide 1004. The guide 1004 is a groove or a channel existing in a wall of the outer catheter 124. As discussed previously, an actuator mechanism can provide longitudinal motion to the transducer array 200. The longitudinal motion can cause rotational motion of the transducer array 200 as the notch 1002 travels back and forth in the guide 1004. A direction of motion of the notch is indicated in FIG. 10. The guide 1004 can be any sort of appropriate pattern. Rings of the transducer array 200 can be fired at different longitudinal positions, and rotation of the rings may provide advantages in the transmission and collection of echo signals. For example, transducers 202 of one ring may fire at different regions of substantially the same longitudinal position than transducers of other rings.

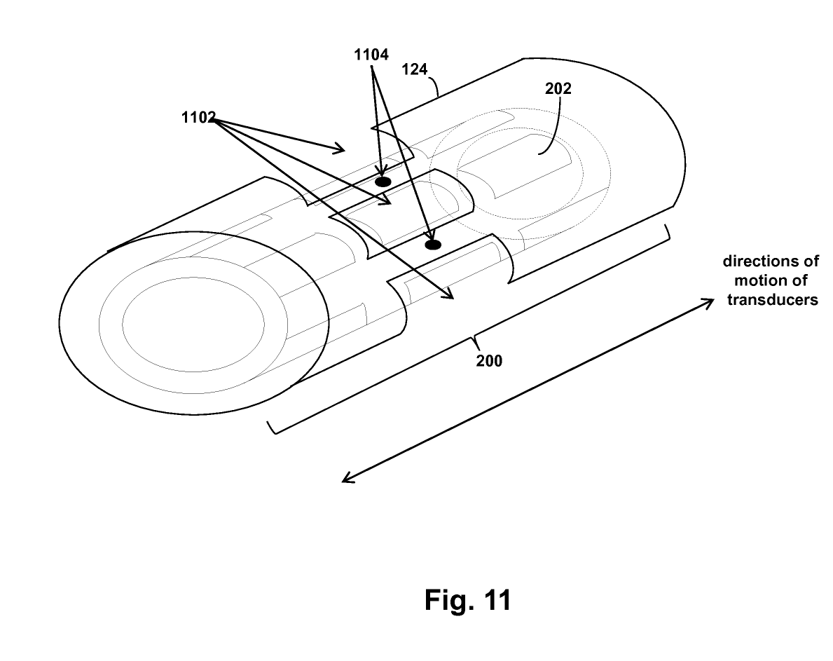

FIG. 11 is a longitudinal perspective view of an ultrasound transducer array 200 and an outer catheter 124 according to an embodiment of the present disclosure. The outer catheter 124 comprises windows 1102 and ultrasound markers 1104. The windows 1102 are substantially transparent to ultrasound signals, so the windows 1102 are portions of the outer catheter that are substantially transparent to ultrasound energy. The windows 1102 can be made of an ultrasound transparent material or the windows 1102 can be cut out such that there is no material between the transducer array 200 and the outside environment. FIG. 11 is not drawn to scale. For example, the outer catheter 124 is illustrated with zero thickness, but the outer catheter 124 in practice will have some thickness. In at least one embodiment, the markers 1104 are affixed to the outer catheter 124.

In an embodiment, a ring of transducers 202 is fired when the ring substantially aligns with the windows 1102. The markers 1104 will show in at least one ultrasound echo. The ultrasound markers 1104 can be used by a position control system analyzing the return echoes to align transducers 202 with windows 1102. In an alternative form, the markers present in the return data can be used in signal processing to determine the position that a ring of transducers 202 fired from in order to correct for any misalignments or offsets of transducer firings.

Ultrasound markers can be used with any type of outer catheter. For example, ultrasound markers can also be used with continuous outer catheters, such as those illustrated in the regions 900 and 1000. The markers used with continuous outer catheters can be used as described above with respect to FIG. 11. That is, one or more markers will show in at least one transducer echo. Software can then analyze the echoes to ensure the rings of transducers are aligned at firing by controlling an actuator or through processing can take into account slight offsets in firing position among the different rings.

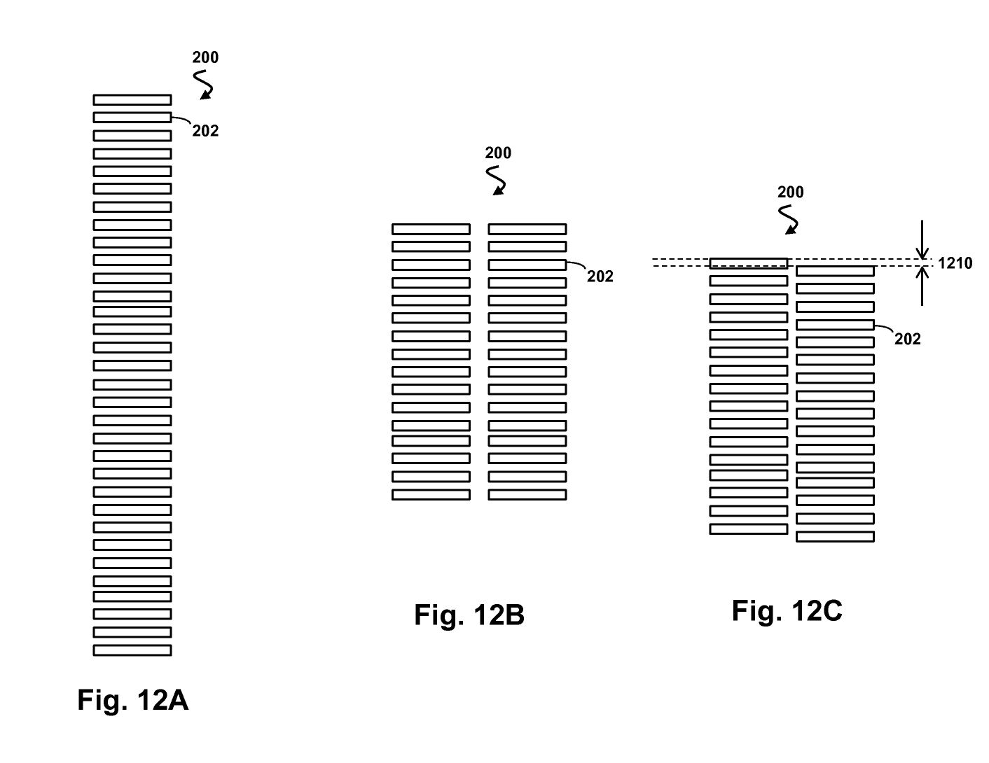

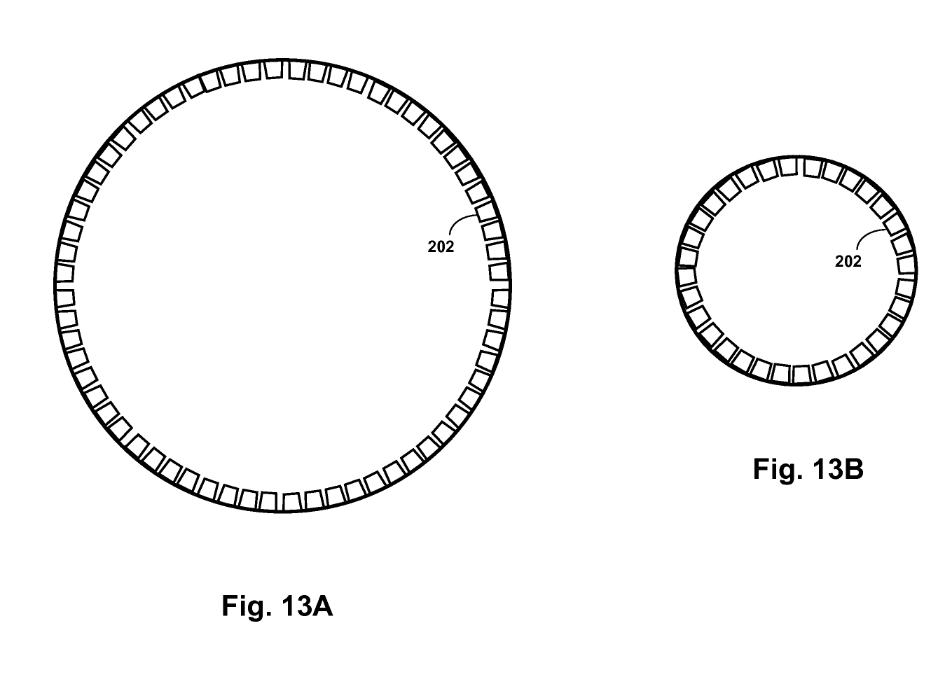

FIGS. 12A-12C are top views of ultrasound transducer arrays 200 depicted in their flat form according to various embodiments of the present disclosure. FIG. 12A illustrates one ring or circumferential array of thirty-two transducers 202. FIG. 12B illustrates two rings of sixteen transducers 202 for a total of thirty-two transducers. FIG. 12C also illustrates two rings of sixteen transducers 202 for a total of thirty-two transducers, with one ring being rotationally offset from the other ring by an amount indicated by 1210. FIGS. 12A-12C thus illustrate different configurations of thirty-two transducers 202. The configurations in FIGS. 12B and 12C have an advantage over the configuration of FIG. 12A in that when the transducer arrays 200 are folded into a cylindrical shape the configurations in FIGS. 12B and 12C have a much smaller diameter than the configuration in FIG. 12A. FIGS. 13A and 13B illustrate this point for 64 transducers 202. There is a second ring of thirty-two transducers 202 behind the first ring of transducers 202 shown in FIG. 13B for a total of sixty-four transducers 202. Although the cross-sectional views are not necessarily drawn to scale, the views illustrate that using multiple rings of transducers 202 allow for a smaller diameter transducer array for a given number of transducers than using a single ring. Such arrangements can more easily satisfy space constraints associated with lumen and/or catheter dimensions.

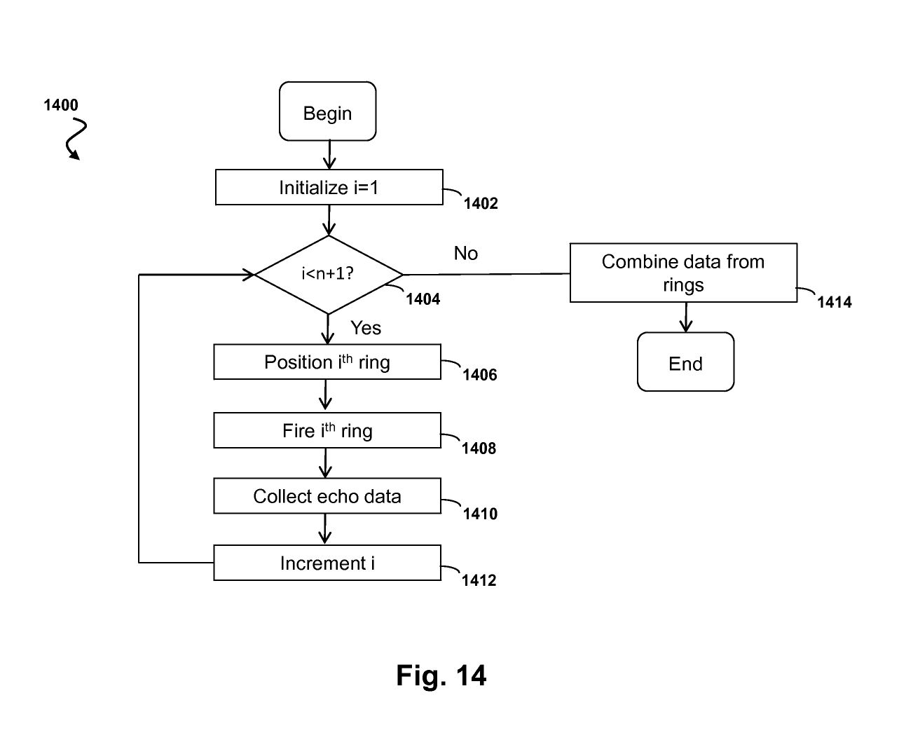

FIG. 14 is a flow diagram of a method 1400 of generating ultrasound data according to aspects of the present disclosure. The method 1400 is described with reference to FIG. 14 and other figures described previously. The method includes a “for” loop in which an index “i” is initialized as i=1 in block 1402. The index represents a ring or circumferential array in a transducer array. If the condition in block 1404 is met, i.e., if i<n+1, where n is a number of rings in the transducer array. In block 1406, the i^{th} ring is positioned. A ring can be positioned using an actuator mechanism 504. Example actuator mechanisms 504 are described herein. In block 1408, transducers in the i^{th} ring are fired or emit an ultrasound signal. In an embodiment, one transducer on at a time on the i^{th} ring emits an ultrasound signal and one or more transducers on the ring receive ultrasound echoes. Data is collected in block 1410 from echoes from different ultrasound signals emitted by a ring. Thus, blocks 1408 and 1410 can be performed in alternating fashion for each ring. For example, in an embodiment there are four rings of thirty-two transducers per ring. For each ring, one transducer fires and one or more other transducers on the ring receive echoes. Then another transducer on the ring fires, and one or more other transducers on the ring receive echoes. This process is repeated until all thirty-two transducers on a ring fire. In block 1412, the index “i” is incremented and the condition in block 1404 is determined. Blocks 1406-1412 are repeated for each ring until echo data is collected for each ring. In addition to the firing and collection sequence described above, or in place of that technique, the system may utilize transducers in other longitudinally spaced rings to collect echo data. For example, a transducer in the i^{th} ring may be fired with a transducer in the ith+1 ring listening for the echo. To increase the available data for processing, the technique may also include listening for echo returns with a transducer in the ith+2 ring. While the foregoing description has been simplified for the purpose of explanation, it will be appreciated that echo information collected from more than one transducer may be combined to create a composite ultrasound image. Such techniques are disclosed in U.S. patent application Ser. No. 13/974,757, published as U.S. Patent Application Publication No. 2014/0056099 on Feb. 27, 2014 which is hereby incorporated by reference in its entirety. The images of the ultrasound echo data can be displayed in a tomographic or other image suitable for display on a monitor or other viewing device.

The method 1400 provides for a variety of combinations of longitudinal transducer arrays and firing positions. In some embodiments, each ring fires in substantially the same position. For example, FIGS. 8A-8C illustrate such an arrangement for rings that are longitudinally aligned. In another embodiment, each ring fires in substantially the same position but at least one ring is rotationally offset from the others, for example as shown in FIG. 4. In still another aspect, one firing sequence includes a front transducer firing with a transducer on the middle ring listening. In a further aspect, in this sequence as an optional feature, a transducer on the third transducer ring also listens for the echo of the front transducer. The sequence can continue until all of the transducers on the front ring have been fired.

Various embodiments may add, omit, rearrange, or modify the actions of method 1400. For example, in some embodiments involving a transducer array with a plurality of rings, the transducer array is stationary. In embodiments with a stationary transducer array, block 1406 can be omitted. Data is collected from each ring, but the rings fire without changing positions of various rings.

In block 1414, data from all of the rings is combined to generate an image. By combining echo data from different rings imaging performance can be improved as compared to using data from only one ring. For example, if each ring fires in substantially the same longitudinal position, data collected from each ring can be averaged during processing to reduce effects of noise from any one transducer. The IVUS console 106 aggregates and can assemble the received echo data to create an image of vascular structure for display on the monitor 108.

As another example, data collected from each ring can be combined using a weighted average to emphasize data from one ring over another. Such an arrangement may be beneficial if each ring is tuned to or designed to fire at a different ultrasonic frequency (e.g., using rings of varying thicknesses). The depth of field of an ultrasonic signal varies with frequency, with depth of field decreasing as frequency increases. When an ultrasonic signal is transmitted echoes are received from features at various distances. Distances to objects can be measured based on the time a signal is emitted from a transducer until an echo is received, and this time can be referred to as the round-trip time. Echo's generated by rings tuned to different frequencies can be weighted differently depending on the desired resolution and depth of field. In various embodiments, image processing weights return data such that longer round-trip echoes are given more weight in the image from lower frequencies while shorter round-trip echoes are given more weight for higher frequencies to achieve the benefits of the different ultrasonic frequencies. For each ultrasound signal, the weighting can vary as a function of round-trip travel time. For example, for signals with relatively high ultrasound frequency, the weighting of echo data decreases versus round-trip travel time. In an embodiment, a longitudinal transducer array comprises four rings, with a first ring tuned or designed to 10 MHz, a second ring tuned or designed to 20 MHz, a third ring tuned or designed to 40 MHz, and a fourth ring tuned or designed to 60 MHz.

In another example embodiment, suppose a transducer array is employed that comprises a plurality of rings with at least one ring tuned to a different frequency than another one of the rings. Instead of collecting data from all of the rings, only one ring can be selected to fire. The firing ring can be selected on the basis of the desired depth of field.

Current transducer arrays are limited in the number of transducers used due to space constraints associated with lumen and/or catheter dimensions and manufacturing processes used to generate individual elements. By placing circumferential arrays in offset longitudinal configurations and oscillating the arrays forward and backward, a larger number of elements can be utilized without compromising the transducer outer diameter. Software can be utilized to generate an image using all of elements for a fixed location along the length array, utilizing a time based algorithm. Alternatively, a same number of transducer elements can be employed as a conventional array but in a reduced device profile, which can lead to improved manufacturability.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging device, comprising:
a flexible elongate member; and
an ultrasound scanner assembly disposed at a distal portion of the flexible elongate member, wherein the ultrasound scanner assembly includes an ultrasound transducer array, wherein the ultrasound transducer array includes a plurality of rings of transducers.

2. The device of claim 1, wherein each of the plurality of rings of transducers comprises a same number of transducers, and wherein the rings of transducers are substantially in longitudinal alignment.

3. The device of claim 1, wherein each of the plurality of rings of transducers comprises a same number of transducers, and wherein at least two of the plurality of rings of transducers are helically offset from each other.

4. The device of claim 1, wherein at least one of the plurality of rings operates at a different ultrasound frequency than another one of the plurality of rings.

5. The device of claim 1, wherein the ultrasound scanner assembly further comprises an actuator mechanism, wherein the actuator mechanism comprises an actuator and an anchor, wherein the anchor is secured relative to the elongate member, wherein the actuator is connected to the transducer array and configured to move the transducer array.

6. The device of claim 5, wherein the actuator is a shape memory alloy (SMA) actuator having an activated and a deactivated state, wherein the SMA actuator and the transducer array move along the longitudinal axis of the elongate member upon activation of the SMA actuator.

7. The device of claim 5, further comprising an outer catheter, wherein the ultrasound scanner assembly is located inside of the outer catheter.

8. The device of claim 7, wherein the longitudinal axis of the outer catheter is substantially aligned with the longitudinal axis of the ultrasound scanner assembly.

9. The device of claim 5, further comprising an outer catheter that circumscribes at least a portion of the ultrasound scanner assembly.

10. The device of claim 8, wherein the outer catheter comprises a plurality of windows substantially in circular alignment.

11. The device of claim 10, further comprising at least one ultrasound marker positioned relative to a window such that the at least one ultrasound marker will appear in a transducer echo.

12. The device of claim 8, wherein the outer catheter comprises a guide channel, wherein the scanner assembly comprises a notch that fits within the guide channel such that upon activation of the actuator the scanner assembly moves along the longitudinal axis of the elongate member and rotates about the longitudinal axis.

13. The device of claim 8, wherein the outer catheter is a continuous outer catheter, wherein the device further comprises a lubricious fluid disposed between the outer catheter and a portion of the ultrasound scanner assembly.

14. The device of claim 13, wherein the lubricious fluid is impedance matched to at least one of the transducer, the matching layer or the outer sheath for ultrasound transmission.

15. The device of claim 1, wherein the plurality of rings of transducers comprises two, three, or four rings of transducers with each ring comprising 16 or more transducers.

16. A method of ultrasound imaging using an intravascular ultrasound (IVUS) device, wherein the device comprises an ultrasound scanner assembly including an ultrasound transducer array, and wherein the ultrasound transducer array includes a plurality of rings of transducers, the method comprising:
for each of the plurality of rings, performing the following:
emitting an ultrasonic waveform by at least one transducer in the corresponding ring;
generating echo data by the ultrasound scanner assembly based on a reflected echo of the ultrasonic waveform; and
providing the echo data to an intravascular ultrasound (IVUS) console; and processing the echo data generated by each of the plurality of rings for display.

17. The method of claim 16, further comprising:
positioning each ring at substantially the same longitudinal position for emitting the corresponding ultrasonic waveform.

18. The method of claim 17, wherein generating echo data comprises:
using each ring that emitted an ultrasonic waveform to receive the corresponding reflected echo and generate the echo data.

19. The method of claim 17, wherein generating echo data comprises:
using a different ring to receive the reflected echo and generate the echo data than the ring that emitted the ultrasonic waveform.

20. The method of claim 17, wherein processing the echo data comprises:
weighting the echo data from at least two transducer rings by a corresponding weighting factor and combining the weighted echo data from at least two transducer rings.

21. The method of claim 20, wherein at least one transducer ring is tuned to a different ultrasonic frequency than another one of the transducer rings.

22. The method of claim 17, wherein the positioning comprises controlling an actuator mechanism coupled to the ultrasound transducer array to position each ring.

23. The method of claim 22, wherein the device further comprises an outer catheter that circumscribes at least a portion of the ultrasound scanner assembly, wherein the outer catheter comprises a plurality of windows substantially in circular alignment and at least one ultrasound marker that affects echo data, wherein each ring is positioned using ultrasound marker characteristics in the echo data.

24. The method of claim 16, wherein the ultrasound transducer array remains in substantially the same position while emitting the ultrasonic waveforms.
